(19) United States
(12) Patent Application Publication
(10) Pub. No.: US 2025/0057805 A1
(43) Pub. Date: Feb. 20, 2025

(54) METHODS FOR ADDRESSING INJECTION SITE REACTIONS ASSOCIATED WITH THE ADMINISTRATION OF BEVEMIPRETIDE

(71) Applicant: Stealth BioTherapeutics Inc., Needham, MA (US)

(72) Inventors: Anthony Abbruscato, Needham, MA (US); Alana W. Sullivan, Clayton, NC (US); Laura Kropp, Needham, MA (US)

(73) Assignee: Stealth BioTherapeutics Inc., Needham, MA (US)

(21) Appl. No.: 18/819,188

(22) Filed: Aug. 29, 2024

Related U.S. Application Data
(63) Continuation of application No. PCT/US2024/030940, filed on May 24, 2024.
(60) Provisional application No. 63/468,900, filed on May 25, 2023.

(51) Int. Cl. A61K 31/353; A61K 31/138; A61K 31/436; A61K 31/58; A61K 45/06; A61P 37/08

(52) U.S. Cl. CPC A61K 31/353; A61K 31/138; A61K 31/436; A61K 31/58; A61K 45/06; A61P 37/08

(57) ABSTRACT

The present disclosure provides methods for treating, preventing, ameliorating, inhibiting or delaying the onset of injection site reactions associated with the subcutaneous administration of bevemipretide, or a pharmaceutically acceptable salt thereof. The methods may involve administration of inhibitors of the MRGPRX2 receptor and/or inhibitors of mast cell degranulation. In some cases, the methods involve administration of an effective amount of a flavonoid, a coumarin, a phenol, a terpenoid, mometasone furoate, tacrolimus, quercetin, diphenhydramine and/or ice.

### CROSS-REFERENCE TO RELATED APPLICATIONS

This application claims the benefit of and priority to U.S. Provisional Patent Application No. 63/468,900, filed May 25, 2023, the contents of which are incorporated herein by reference in their entirety.

### TECHNICAL FIELD

The present technology relates generally to methods for treating, preventing, ameliorating, inhibiting or delaying the onset of injection site reactions (ISRs) associated with the administration of bevemipretide, or a pharmaceutically acceptable salt thereof.

### INTRODUCTION

Bevemipretide is being developed for use in the treatment of subjects afflicted with amyotrophic lateral sclerosis (ALS), Parkinson's disease (PD), PD with dementia, dementia with Lewy bodies, Multiple System Atrophy, Frontotemporal Lobar Degeneration (FTLD) and other disease where TDP-43, Tau protein and α-synuclein are associated with the disease pathology. Bevemipretide is also being considered for use in the treatment of subjects afflicted with diabetic macular edema, macular degeneration, (wet or dry) age-related macular degeneration, glaucoma, diabetic retinopathy or retinitis pigmentosa.

### SUMMARY

In one aspect, the disclosure of the present technology provides a method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with an effective amount of an inhibitor of a MRGPRX2 receptor.

In some embodiments, contacting the bevemipretide injection site or intended injection site with the inhibitor of the MRGPRX2 receptor treats, prevents, ameliorates, inhibits or delays the onset of mast cell degranulation in the subject.

In some embodiments, the method comprises contacting the bevemipretide injection site or intended injection site with an effective amount of a flavonoid, a coumarin, a phenol or a terpenoid as the inhibitor of a MRGPRX2 receptor (See: Finn, D. F. and Walsh, J. J., Twenty-first century mast cell stabilizers, (2013) 170: 23-37). In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the bevemipretide injection site or intended injection site is contacted with the flavonoid, the coumarin, the phenol or the terpenoid prior to administration of bevemipretide, or a pharmaceutically acceptable salt thereof. In some embodiments, the bevemipretide injection site or intended injection site is contacted with the flavonoid, the coumarin, the phenol or the terpenoid after administration of bevemipretide, or a pharmaceutically acceptable salt thereof. In some embodiments, the bevemipretide injection site or intended injection site is contacted with the flavonoid, the coumarin, the phenol or the terpenoid simultaneously with administration of bevemipretide, or a pharmaceutically acceptable salt thereof.

In some embodiments, the method comprises contacting the bevemipretide injection site or intended injection site with an effective amount of mometasone furoate, tacrolimus, quercetin, diphenhydramine and/or ice.

In some embodiments, mometasone furoate ointment is applied to the injection site to thereby contact the bevemipretide injection site or intended injection site with mometasone furoate. In some embodiments, tacrolimus ointment or quercetin ointment is applied to the injection site to thereby contact the bevemipretide injection site or intended injection site with tacrolimus or quercetin.

In some embodiments, diphenhydramine or quercetin is administered systemically, optionally via oral administration, to the subject to thereby contact the bevemipretide injection site or intended injection site with diphenhydramine or quercetin.

In some embodiments, ice is applied to the injection site or intended injection site to thereby contact the bevemipretide injection site or intended injection site with the ice.

In some embodiments, the bevemipretide injection site or intended injection site is contacted with the mometasone furoate, tacrolimus, quercetin, diphenhydramine and/or ice prior to administration of bevemipretide, or a pharmaceutically acceptable salt thereof. In some embodiments, the bevemipretide injection site or intended injection site is contacted with the mometasone furoate, tacrolimus, quercetin, diphenhydramine and/or ice after administration of bevemipretide, or a pharmaceutically acceptable salt thereof.

In some embodiments, about 5 mg to about 60 mg of bevemipretide, or a pharmaceutically acceptable salt thereof, is subcutaneously administered to the bevemipretide injection site.

In one aspect, the disclosure of the present technology provides a method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with an effective amount of an inhibitor of mast cell degranulation.

In some embodiments, the method comprises contacting the bevemipretide injection site or intended injection site with an effective amount of a flavonoid, a coumarin, a phenol or a terpenoid as the inhibitor of mast cell degranulation. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen- 1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the bevemipretide injection site or intended injection site is contacted with the flavonoid, the coumarin, the phenol or the terpenoid prior to administration of bevemipretide, or a pharmaceutically acceptable salt thereof. In some embodiments, the bevemipretide injection site or intended injection site is contacted with the flavonoid, the coumarin, the phenol or the terpenoid after administration of bevemipretide, or a pharmaceutically acceptable salt thereof. In some embodiments, the bevemipretide injection site or intended injection site is contacted with the flavonoid, the coumarin, the phenol or the terpenoid simultaneously with administration of bevemipretide, or a pharmaceutically acceptable salt thereof.

In some embodiments, the method comprises contacting the bevemipretide injection site or intended injection site with an effective amount of mometasone furoate, tacrolimus, quercetin, diphenhydramine and/or ice.

In some embodiments, mometasone furoate ointment is applied to the injection site or intended injection site to thereby contact the bevemipretide injection site with mometasone furoate. In some embodiments, tacrolimus ointment or quercetin ointment is applied to the injection site or intended injection site to thereby contact the bevemipretide injection site with tacrolimus or quercetin.

In some embodiments, diphenhydramine or quercetin is administered systemically, optionally via oral administration, to the subject to thereby contact the bevemipretide injection site or intended injection site with diphenhydramine or quercetin.

In some embodiments, ice is applied to the injection site or intended injection site to thereby contact the bevemipretide injection site with the ice.

In some embodiments, the bevemipretide injection site or intended injection site is contacted with the mometasone furoate, tacrolimus, quercetin, diphenhydramine and/or ice prior to administration of bevemipretide, or a pharmaceutically acceptable salt thereof. In some embodiments, the bevemipretide injection site or intended injection site is contacted with the mometasone furoate, tacrolimus, quercetin, diphenhydramine and/or ice after administration of bevemipretide, or a pharmaceutically acceptable salt thereof.

In some embodiments, about 5 mg to about 60 mg of bevemipretide, or a pharmaceutically acceptable salt thereof, is subcutaneously administered to the bevemipretide injection site.

In one aspect, the disclosure of the present technology provides a method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with a flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the bevemipretide injection site or intended injection site is contacted with the flavonoid, the coumarin, the phenol or the terpenoid prior to administration of bevemipretide, or a pharmaceutically acceptable salt thereof. In some embodiments, the bevemipretide injection site or intended injection site is contacted with the flavonoid, the coumarin, the phenol or the terpenoid after administration of bevemipretide, or a pharmaceutically acceptable salt thereof. In some embodiments, the bevemipretide injection site or intended injection site is contacted with the flavonoid, the coumarin, the phenol or the terpenoid simultaneously with administration of bevemipretide, or a pharmaceutically acceptable salt thereof.

In some embodiments, about 5 mg to about 60 mg of bevemipretide, or a pharmaceutically acceptable salt thereof, is subcutaneously administered to the injection site.

In one aspect, the disclosure of the present technology provides a method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with mometasone furoate, tacrolimus, quercetin, diphenhydramine and/or ice.

In some embodiments, the bevemipretide injection site or intended injection site is contacted with mometasone furoate, tacrolimus, quercetin, diphenhydramine and/or ice prior to administration of bevemipretide, or a pharmaceutically acceptable salt thereof. In some embodiments, the bevemipretide injection site or intended injection site is contacted with mometasone furoate, tacrolimus, quercetin, diphenhydramine and/or ice after administration of bevemipretide, or a pharmaceutically acceptable salt thereof.

In some embodiments, mometasone furoate ointment is applied to the injection site or intended injection site to thereby contact the bevemipretide injection site with the mometasone furoate ointment. In some embodiments, tacrolimus ointment is applied to the injection site or intended injection site to thereby contact the bevemipretide injection site with tacrolimus ointment or quercetin ointment.

In some embodiments, diphenhydramine or quercetin is administered systemically to the subject to thereby contact the bevemipretide injection site or intended injection site with the diphenhydramine or quercetin.

In some embodiments, ice is applied to the injection site or intended injection site to thereby contact the bevemipretide injection site with the ice.

In some embodiments, about 5 mg to about 60 mg, of bevemipretide, or a pharmaceutically acceptable salt thereof, is subcutaneously administered to the subject. In some embodiments, about 5 mg of bevemipretide, or a pharmaceutically acceptable salt thereof, is subcutaneously administered to the subject. In some embodiments, about 10 mg of bevemipretide, or a pharmaceutically acceptable salt thereof, is subcutaneously administered to the subject. In some embodiments, about 20 mg of bevemipretide, or a pharmaceutically acceptable salt thereof, is subcutaneously administered to the subject. In some embodiments, about 30 mg of bevemipretide, or a pharmaceutically acceptable salt thereof, is subcutaneously administered to the subject. In some embodiments, about 40 mg of bevemipretide, or a pharmaceutically acceptable salt thereof, is subcutaneously administered to the subject. In some embodiments, about 50 mg of bevemipretide, or a pharmaceutically acceptable salt thereof, is subcutaneously administered to the subject. In some embodiments, about 60 mg of bevemipretide, or a pharmaceutically acceptable salt thereof, is subcutaneously administered to the subject.

In one aspect, the disclosure of the present technology provides a method comprising: a) subcutaneously administering an effective amount of bevemipretide, or a pharmaceutically acceptable salt thereof, to a subject; and b) also administering to said subject an inhibitor of a MRGPRX2 receptor and/or inhibitor of mast cell degranulation; wherein steps a) and b) can be performed in either order or simultaneously.

In some embodiments, step (a) is performed prior to performing step (b). In some embodiments, step (b) is performed prior to performing step (a). In some embodiments, step (a) and step (b) are performed simultaneously or substantially simultaneously.

In some embodiments, the subject is administered an inhibitor of the MRGPRX2 receptor. In some embodiments, the subject is administered an inhibitor of mast cell degranulation. In some embodiments, the inhibitor of the MRGPRX2 receptor or the inhibitor of mast cell degranulation is a flavonoid, a coumarin, a phenol or a terpenoid is administered to the subject. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin(3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, an effective amount of mometasone furoate, tacrolimus, quercetin, diphenhydramine and/or ice is administered to the subject as the inhibitor of the MRGPRX2 receptor or the inhibitor of mast cell degranulation.

In some embodiments, the subject is human.

In some embodiments, about 5 mg to about 60 mg of bevemipretide, or a pharmaceutically acceptable salt thereof, is subcutaneously administered to the subject. In some embodiments, about 5 mg of bevemipretide, or a pharmaceutically acceptable salt thereof, is subcutaneously administered to the subject. In some embodiments, about 10 mg of bevemipretide, or a pharmaceutically acceptable salt thereof, is subcutaneously administered to the subject. In some embodiments, about 20 mg of bevemipretide, or a pharmaceutically acceptable salt thereof, is subcutaneously administered to the subject. In some embodiments, about 40 mg of bevemipretide, or a pharmaceutically acceptable salt thereof, is subcutaneously administered to the subject. In some embodiments, about 50 mg of bevemipretide, or a pharmaceutically acceptable salt thereof, is subcutaneously administered to the subject. In some embodiments, about 60 mg of bevemipretide, or a pharmaceutically acceptable salt thereof, is subcutaneously administered to the subject.

In some embodiments, the subject has been diagnosed with diabetic macular edema, macular degeneration, age-related macular degeneration, glaucoma, diabetic retinopathy or retinitis pigmentosa. In some embodiments, the subject has been diagnosed as having age-related macular degeneration (AMD). In some embodiments, the subject diagnosed as having AMD, has drusen. In some embodiments, the subject diagnosed as having AMD, with or without drusen, has been diagnosed with geographic atrophy (GA).

In some embodiments, the subject has been diagnosed with amyotrophic lateral sclerosis (ALS), Parkinson's disease (PD), PD with dementia, dementia with Lewy bodies, Multiple System Atrophy, Frontotemporal Lobar Degeneration (FTLD) or other disease where TDP-43, Tau protein and α-synuclein are associated with the disease pathology.

### BRIEF DESCRIPTION OF THE DRAWINGS

FIG. 1 is an illustration of receptor pathways and common ligands associated with mast cell activation.

FIG. 2 is an illustration of the chemical structure of bevemipretide and its pharmaceutically acceptable, tris-HCl salt.

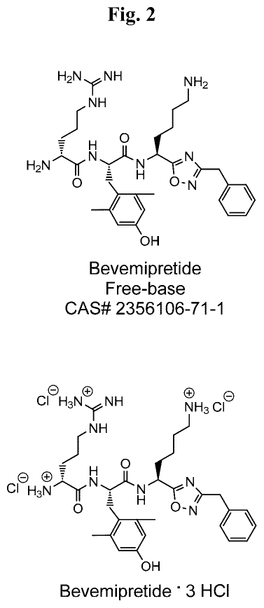

FIG. 3 is a diagram/illustration showing how and where bevemipretide and the various interventions can be administered subcutaneously to human subjects.

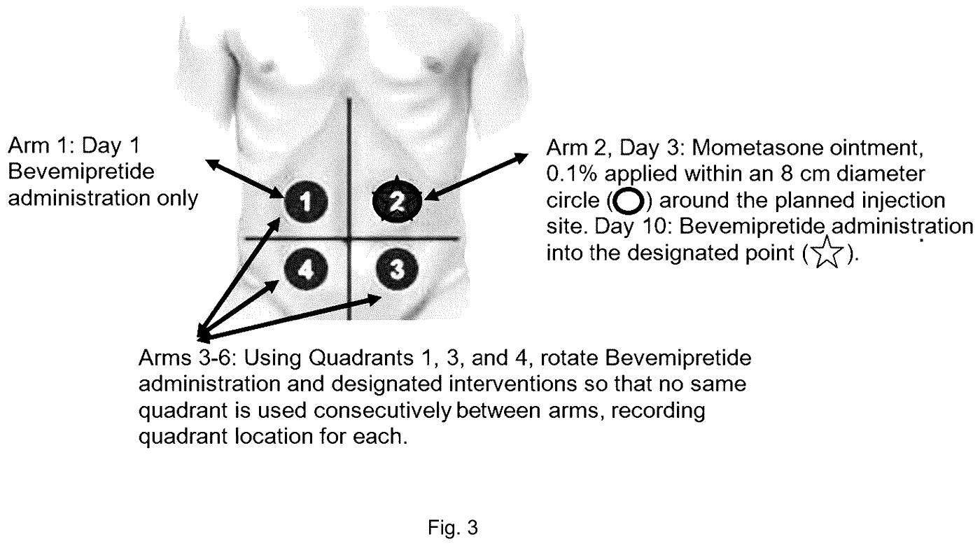

FIG. 4 is a graphic illustration of the responses of MRGPRX2-expressing HEK293 cells to bevemipretide at the indicated concentrations to generate an EC50.

FIG. 5A is a graphic illustration of the treatment of a human cell line that stably expresses the MRGPRX2 receptor with various concentrations of bevemipretide showing that a dose-dependent immune response is elicited by bevemipretide. FIG. 5B is a graphic illustration of the treatment of the parent human cell line used in FIG. 5A, but which lacks the MRGPRX2 receptor, with various concentrations of bevemipretide thereby demonstrating that there is no immune response in this control cell line lacking the MRGPRX2 receptor. Ionomycin is a membrane-permeable calcium ionophore. It increases intracellular calcium levels in a manner that is not dependent on the presence of the MRGPRX2 receptor.

FIG. 6 is a graphic illustration of the treatment of the human cell line known to express the MRGPRX2 receptor (i.e. the cell line used in FIG. 5A) with Ionomycin (a positive control), Leuprolide (a known agonist of the MRGPRX2 receptor) and DPBS (a negative/vehicle control).

FIG. 7A is a graphic illustration of the treatment of the human cell line known to express the MRGPRX2 receptor (i.e. the cell line used in FIG. 5A) with 100 μM of bevemipretide and various concentrations of quercetin, a suspected antagonist of the MRGPRX2 receptor. The data demonstrates that quercetin inhibits activation of MRGPRX2(or more correctly stated as calcium mobilization that results from MRGPRX2 activation) elicited by bevemipretide in a dose-dependent manner. FIG. 7B is a graphic illustration of the treatment of the human cell line known to express the MRGPRX2 receptor (i.e. the cell line used in FIG. 5A) with 100 μM of bevemipretide and various concentrations of mometasone, a suspected antagonist of the MRGPRX2 receptor.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

In a recently completed phase 1 human clinical trial, injection site reactions were observed as an adverse event that resulted from subcutaneous administration of bevemipretide. Similarly, in long term toxicology studies in rats, injection site reactions were observed from subcutaneous administration; some being severe at the higher dose levels administered. Injection site reactions were likewise observed in long term toxicology studies of non-human primates, but these were generally not severe even at the highest dosages tested.

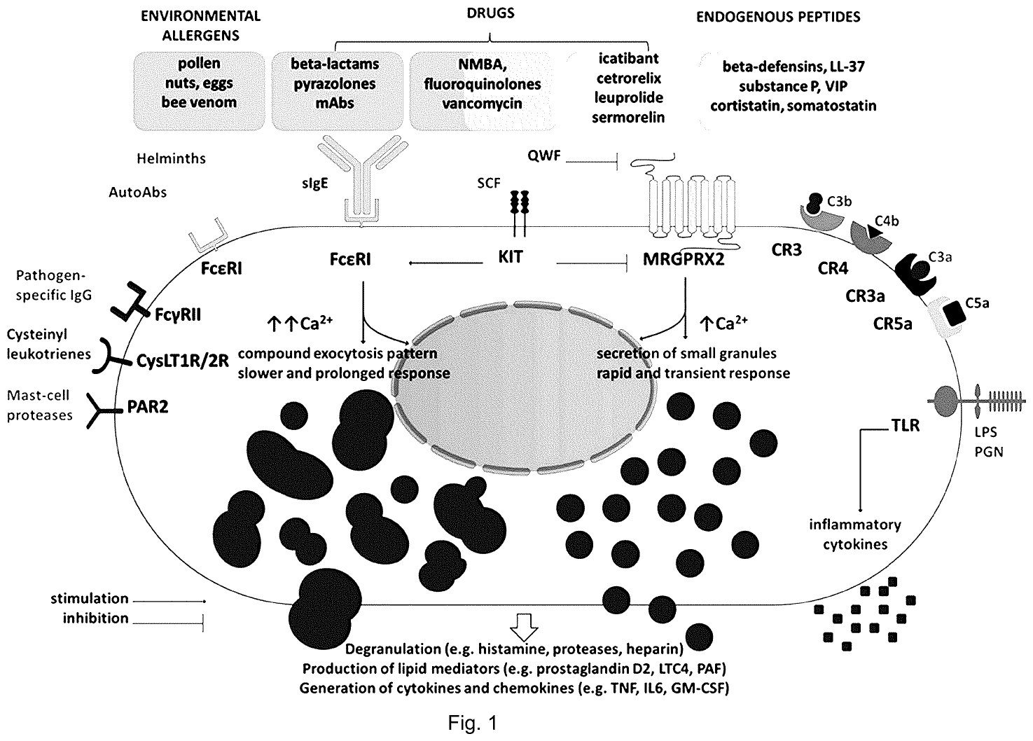

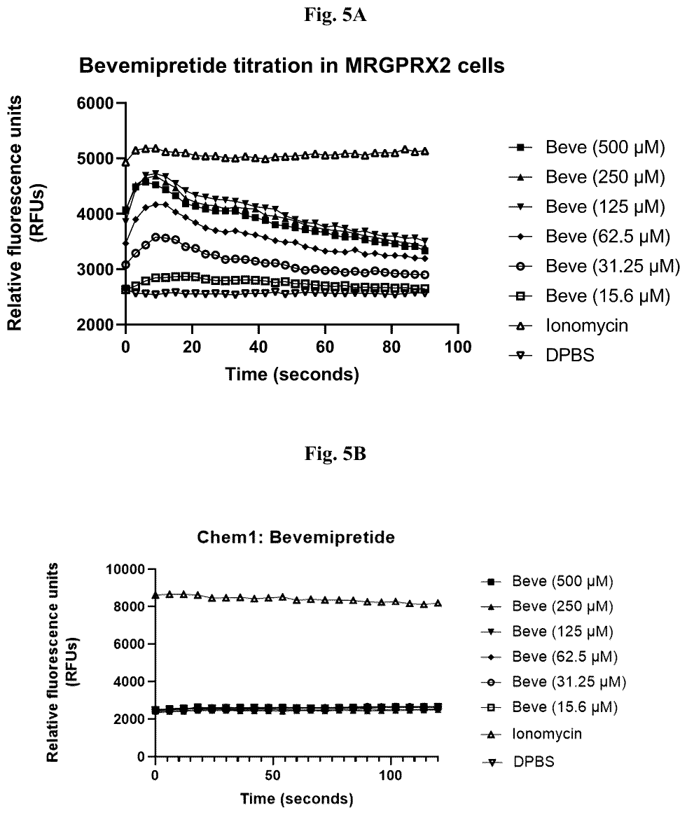

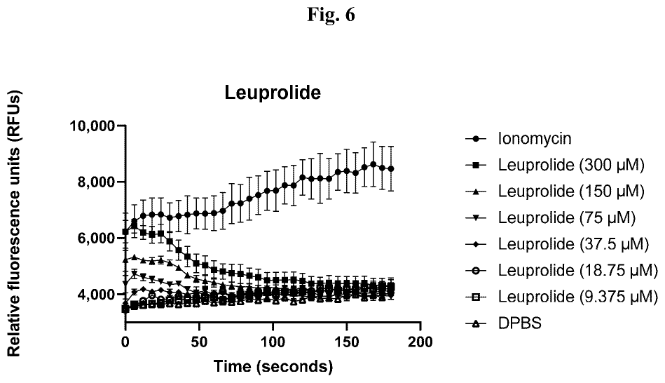

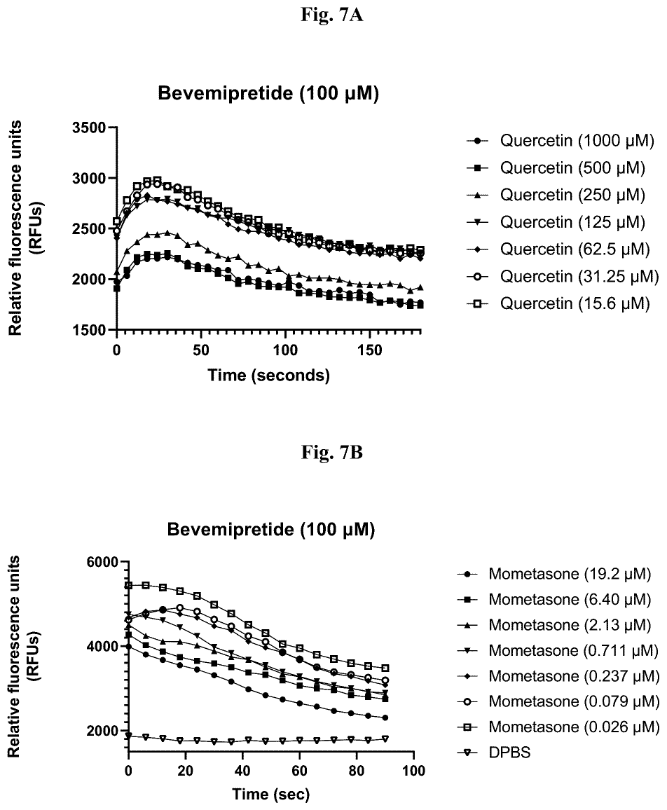

In order to ascertain the basis for these observed injection site reactions, in vitro assays were performed and these demonstrated that bevemipretide activates MRGPRX2, a receptor expressed primarily on skin mast cells (FIG. 1; Example 1). Activation of the MRGPRX2 receptor is known to result in mast cell degranulation and a resulting allergic/inflammatory reaction. In the assays performed, calcium mobilization in response to stimulation with bevemipretide is both MRGPRX2-receptor dependent and dose-dependent (FIG. 5A, FIG. 5B and FIG. 6). The calcium mobilization in response to stimulation with bevemipretide can be inhibited in a dose-dependent manner with quercetin, a suspected antagonist of the MRGPRX2 receptor (FIG. 7). This in vitro data was consistent with the human and animal studies that were conducted and strongly suggests that this pathway is at least partially responsible for the observed inflammatory response at the bevemipretide injection site. Hence, administration of inhibitors of the MRGPRX2-receptor and inhibitors of mast cell degranulation (e.g., quercetin) should prove beneficial to treat, prevent, inhibit, ameliorate and/or delay the onset of the injection site reactions associated with subcutaneous administration of bevemipretide.

### I. Definitions

It is to be appreciated that certain aspects, modes, embodiments, variations and features of the technology are described below in various levels of detail in order to provide a substantial understanding of the present application. The definitions of certain terms as used in this specification are provided below. Unless defined otherwise, all technical and scientific terms used herein generally have the same meaning as commonly understood by one of ordinary skill in the art to which this technology belongs.

As used in this specification and the appended embodiments, the singular forms “a”, “an” and “the” include plural references unless the content clearly dictates otherwise. For example, reference to “a cell” includes a combination of two or more cells, and the like.

As used herein, “administering” or the “administration” of an agent (i.e. therapeutic agent) or compound/drug product (including a composition) to a subject includes any route of introducing or delivering to a subject a compound/drug product to perform its intended function. Administration may be carried out by any suitable route, such as oral administration. Administration can be carried out subcutaneously. Administration includes self-administration, the administration by another or administration by use of a device (e.g., an infusion pump).

As used herein, to “ameliorate” or “ameliorating” a disease, disorder or condition refers to results that, in a statistical sample or specific subject, make the occurrence of the disease, disorder or condition (or a sign, symptom or condition thereof) better or more tolerable in a sample or subject administered a therapeutic agent relative to a control sample or subject.

As used herein the terms “carrier” or “pharmaceutically acceptable carrier” refer to a diluent, adjuvant, excipient, or vehicle with which a compound/drug product/composition (including a medicament) is administered or formulated for administration. Non-limiting examples of such pharmaceutically acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the phrase “contacting the bevemipretide injection site” can also be interpreted as “administering to the bevemipretide injection site” wherein the administration can be direct or indirect.

As used herein, the phrase “delaying the onset of” refers to, in a statistical sample, postponing, hindering the occurrence of a disease, disorder or condition, or causing one or more signs, symptoms or conditions to occur more slowly than normal, in a sample or subject administered a therapeutic agent relative to a control sample or subject.

As used herein, the term “effective amount” refers to a quantity of a compound/composition/drug product sufficient to achieve a desired therapeutic and/or prophylactic effect, e.g., an amount that treats, prevents, inhibits, ameliorates, or delays the onset of the disease, disorder or condition (e.g., an injection site reaction), or the physiological signs, symptoms or conditions of the disease or disorder (e.g., amyotrophic lateral sclerosis (ALS), Parkinson's disease (PD), PD with dementia, dementia with Lewy bodies, Multiple System Atrophy, Frontotemporal Lobar Degeneration (FTLD) and other disease where TDP-43, Tau protein and α-synuclein are associated with the disease pathology, diabetic macular edema, macular degeneration, (wet or dry) age-related macular degeneration, glaucoma, diabetic retinopathy or retinitis pigmentosa, AMD with drusen, or AMD with geographic atrophy (GA)). In the context of therapeutic or prophylactic applications, in some embodiments, the amount of a compound/composition/drug product administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compounds/compositions/drug products can also be administered in combination with one or more additional therapeutic compounds/agents (a so called “co-administration” where, for example, the additional therapeutic agent could be administered simultaneously, sequentially or by separate administration).

As used herein, “bevemipretide” refers to the peptidomimetic of formula:

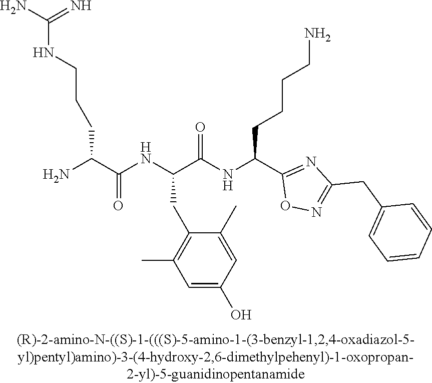 [SMILES: Cc1cc(O)cc(C)c1C[C@H](NC(=O)[C@H](N)CCCNC(=N)N)C(=O)N[C@@H](CCCCN)c1nc(Cc2ccccc2)no1]

Bevemipretide is also referred to in scientific literature as CAS #2356106-71-1 (in its free base form). Bevemipretide can be administered as the pharmaceutically acceptable salt, such as a tris-HCl salt (CAS #2589640-11-7) having the formula:

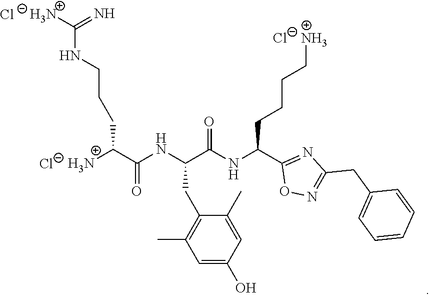 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

Whenever the term, bevemipretide is used herein, its use is intended to also encompass pharmaceutically acceptable salts thereof, unless the context of its use is clearly contradictory to such an interpretation.

As used herein, “inhibit” or “inhibiting” refers to the reduction in injection site reactions as an objectively measurable amount or degree compared to a control. In one embodiment, inhibit or inhibiting refers to the reduction by at least a statistically significant amount compared to a control (or control subject). In one embodiment, inhibit or inhibiting refers to a reduction by at least 5 percent compared to control (or control subject). In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control (or control subject).

As used herein, “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]+). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology

As used herein, “prevention” or “preventing” of a disease, disorder, or condition refers to results that, in a statistical sample, exhibit a reduction in the occurrence of the disease, disorder, or condition in a sample or subject administered a therapeutic agent relative to a control sample or subject. Such prevention is sometimes referred to as a prophylactic treatment.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

As used herein, the terms “treating” or “treatment” refer to therapeutic treatment, wherein the object is to reduce, alleviate or slow down (lessen) a pre-existing disease or disorder, or its related signs, symptoms or conditions. By way of example, but not by way of limitation, a subject is successfully “treated” for a disease if, after receiving an effective amount of the compound/composition/drug product or a pharmaceutically acceptable salt thereof, the subject shows observable and/or measurable reduction in or absence of one or more signs, symptoms or conditions associated with the disease, disorder or condition. It is also to be appreciated that the various modes of treatment of medical conditions as described are intended to mean “substantial,” which includes total alleviation of conditions, signs or symptoms of the disease or disorder, as well as “partial,” where some biologically or medically relevant result is achieved.

### II. Pharmaceutical Compositions, Routes of Administration, and Dosing

Injection site reactions (ISRs) were reported and have been observed in subjects receiving (SQ) administered bevemipretide. Injection site reactions can be a significant reason for clinical study participants to prematurely depart from active clinical trials. As such, methodologies directed to treating, preventing, ameliorating, inhibiting or delaying the onset of ISRs in subjects could lead to more robust outcomes from clinical trials and improved compliance of subjects under a treatment regime involving SQ administration of bevemipretide.

Bevemipretide, or a pharmaceutically acceptable salt thereof, can be administered subcutaneously as a buffered aqueous solution (i.e. preserved, buffered saline) via a needle and syringe. For healthy human adults, SQ injections containing 5 mg to 60 mg (daily) have been administered. In animals, SQ administration has been from 1 mg/kg/day to 30 mg/kg/day. The dose administered to a subject may be varied from patient to patient based on factors such as potency, relative bioavailability, patient body weight, renal impairment, severity of adverse side-effects, toxicity and mode of administration.

Mometasone furoate is a corticosteroid used to treat asthma, allergic rhinitis, nasal congestion, nasal polyps, dermatitis, and pruritus. Mometasone furoate is available under the product names: Asmanex®, Dulera®, Elocom™, Elocon®, Nasonex®, Ryaltris®, Sinuva® and Zenhale®. Mometasone furoate can be obtained in a cream/ointment for topical use or as a nasal spray. Mometasone furoate ointment (0.1% w/w) is commonly used for topical administration for relief of the inflammatory and pruritic manifestations of corticosteroid-responsive dermatoses.

Tacrolimus is sold under the trade name Prograf® as an oral formulation. In this form it is generally used with other medicines to help prevent organ rejection in people who have had a kidney, liver, heart or lung transplant. Tacrolimus is also sold in ointment form (e.g. 0.03 to 0.1% w/w) for topical administration under the tradenames: Protopic®, Tacrolim® or Fougera®) for use in the treatment of T-cell-mediated diseases such as eczema and psoriasis. It is often used to treat these conditions when more conventional treatments fail. Tacrolimus is an immunosuppressant and belongs to a class of drugs known as topical calcineurin inhibitors (TCIs).

Diphenhydramine (more commonly known as Benadryl®) is an orally administered antihistamine that reduces the effects of natural chemical histamine in the body. Diphenhydramine is an antagonist and acts primarily as an inverse agonist of the histamine H₁ receptor. It is available as a liquid suspension as well as in tablet, caplet and capsule forms; all used for oral administration. Diphenhydramine is used to treat sneezing, runny nose, watery eyes, hives, skin rash, itching, and other cold or allergy symptoms. Diphenhydramine is also used to treat motion sickness, to induce sleep, and to treat certain symptoms of Parkinson's disease. As with other medications, diphenhydramine dosing often depends on the patient's age and patient's body weight.

Quercetin is a flavonoid that exhibits antioxidant, anti-inflammatory, antihypertensive, and vasodilator effects/properties. Quercetin is found, inter alia, in onions, grapes, berries, cherries, broccoli and citrus fruit. Quercetin is available in various over-the-counter preparations for oral administration. Quercetin is also available in various over-the-counter preparations for topical administration (i.e., ointment/skin cream forms).

Icing has traditionally been used to treat injuries under the premise that it alleviates pain, reduces tissue metabolism, and modifies vascular responses to decrease swelling. Ice may help reduce redness, swelling, and pain in inflammatory-type pimples (e.g., acne).

As stated above, an “effective amount” refers to any amount of the active compound (or compounds; alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e., preventative) or therapeutic treatment regimen can be planned which does not cause substantial unwanted toxicity and yet is effective to treat the particular condition or disease of a particular subject. The effective amount for any particular indication can vary depending on such factors as the disease, disorder or condition being treated, the particular compound or compounds being administered, the size of the subject, or the severity of the disease, disorder or condition. The effective amount may be determined during pre-clinical trials and/or clinical trials by methods familiar to physicians and clinicians. One of ordinary skill in the art can empirically determine the effective amount of a therapeutic agent(s) without necessitating undue experimentation. A maximum dose may be used, that is, the highest safe dose according to some medical judgment. Multiple doses per day may be contemplated to achieve appropriate systemic levels of compounds.

For any therapeutic agent described herein the therapeutically effective amount can, for example, be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

A therapeutic compound/agent (e.g. bevemipretide or mometasone furoate or tacrolimus, or quercetin or diphenhydramine) disclosed herein can be delivered to the subject in a formulation or medicament (i.e., a pharmaceutical composition). Formulations and medicaments can be prepared by, for example, dissolving or suspending a therapeutic compound/agent disclosed herein in water, a pharmaceutically acceptable carrier, salt, (e.g., NaCl or sodium phosphate), buffering agents, preservatives, compatible carriers, adjuvants, and optionally other therapeutically acceptable ingredients.

The pharmaceutical compositions (e.g., a formulation or medicament) can include a carrier (e.g., a pharmaceutically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Glutathione and other antioxidants can be included to prevent oxidation. In many cases, it will be advantageous to include isotonic agents, for example, sugars (e.g., trehalose), polyalcohols such as mannitol, sorbitol, or sodium chloride in the composition. Prolonged absorption of the injectable compositions can be brought about by including in the composition an agent that delays absorption, for example, aluminum monostearate or gelatin.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and may be preserved against the contaminating action of microorganisms such as bacteria and fungi.

Sterile injectable solutions (e.g., a formulation or medicament) can be prepared by incorporating the active compound (e.g. bevemipretide) in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

In addition to the formulations described above, a therapeutic compound/agent (e.g. bevemipretide) disclosed herein may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

In some embodiments, the therapeutic agent(s) (e.g., bevemipretide) is/are administered as a depot formulation wherein the active therapeutic agent(s) is/are encapsulated by, or disposed within, silica-based microparticles. In some embodiments, the ocular formulation can be injected into the eye, for example as a sol-gel (e.g., a silica sol-gel). In some embodiments, the ocular formulation is a depot formulation such as a controlled release formulation (see below). Such controlled release formulation may comprise particles, such as microparticles or nanoparticles.

The pharmaceutical compositions also may comprise suitable solid or gel-phase carriers or excipients. Examples of such carriers or excipients include but are not limited to calcium carbonate, calcium phosphate, various sugars, starches, cellulose derivatives, gelatin, silica/silicone and polymers such as polyethylene glycols.

The therapeutic agent(s), including specifically but not limited to a therapeutic compound/agent disclosed herein (e.g. bevemipretide), may be provided in particles. Particles as used herein means nanoparticles or microparticles (or in some instances larger particles) which can consist in whole or in part of the therapeutic compound/agent or the other therapeutic agent(s) as described herein. The particles may contain the therapeutic compound(s)/agent(s) (e.g., bevemipretide) in a core surrounded by a coating, including, but not limited to, an enteric coating. The therapeutic compound(s)/agent(s) also may be dispersed throughout the particles. The therapeutic compound(s)/agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic compound(s)/agent(s), any of those materials routinely used in the art of pharmacy and medicine, including, but not limited to, erodible, non-erodible, biodegradable, or nonbiodegradable material or combinations thereof. The particles may be microcapsules which contain the therapeutic compound(s)/agent(s) in a solution or in a semi-solid state. The particles may be of virtually any shape.

Both non-biodegradable and biodegradable polymeric materials can be used in the manufacture of particles for delivering the therapeutic compound(s)/agent(s) (e.g., bevemipretide). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, polyethylene glycols (PEGs), polyvinylalcohols (PVAs), poly(methyl methacrylates), poly(ethyl methacrylates), poly(butylmethacrylate), poly(isobutyl methacrylate), poly-lactic acid (PLA), poly(lactic-co-glycolic) acid (PLGA), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and poly(ε-caprolactone) or mixtures of two or more of the foregoing.

Therapeutic compound(s)/agent(s) (e.g. bevemipretide) or mixtures thereof can be formulated in a carrier system. The carrier can be a colloidal system. The carrier or colloidal system can be a liposome, a phospholipid bilayer vehicle. In one embodiment, therapeutic compound(s)/agent(s) or mixtures thereof can be encapsulated in a liposome while maintaining integrity of the therapeutic compound(s)/agent(s) or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound (e.g., bevemipretide) or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In some embodiments, the polymer is poly-lactic acid (PLA), poly lactic/glycolic acid (PLGA) or a mixture thereof. The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the nanoparticles or microparticles can be silica-based or silane-based (See for example: WO2002/080977 entitled: “Biodegradable carrier and method for preparation thereof”).

In some embodiments, the therapeutic compound(s)/agent(s) (e.g. bevemipretide) or mixtures thereof are prepared with carriers that will protect the therapeutic compound(s)/agent(s) or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compound(s)/agent(s) (e.g. bevemipretide) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom to thereby make it available to the subject. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g. a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient(s) for at least 2 days. In some embodiments, the implant or depot formulation is constructed and arranged to deliver therapeutic or prophylactic levels of the active ingredient(s) for at least 7 days. In some embodiments, the implant or depot formulation is constructed and arranged to deliver therapeutic or prophylactic levels of the active ingredient(s) for at least 14 days. In some embodiments, the implant or depot formulation is constructed and arranged to deliver therapeutic or prophylactic levels of the active ingredient(s) for at least 30 days. In some embodiments, the implant or depot formulation is constructed and arranged to deliver therapeutic or prophylactic levels of the active ingredient(s) for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient(s) for at least 180 days. In some embodiments, the implant or depot formulation is constructed and arranged to deliver therapeutic or prophylactic levels of the active ingredient(s) for at least one year. In some embodiments, the implant or depot formulation is constructed and arranged to deliver therapeutic or prophylactic levels of the active ingredient(s) for 15 to 30 days. In some embodiments, the implant or depot formulation is constructed and arranged to deliver therapeutic or prophylactic levels of the active ingredient(s) for 30 to 60 days. In some embodiments, the implant or depot formulation is constructed and arranged to deliver therapeutic or prophylactic levels of the active ingredient(s) for 60 to 90 days. In some embodiments, the implant or depot formulation is constructed and arranged to deliver therapeutic or prophylactic levels of the active ingredient(s) for 90 to 120 days. In some embodiments, the implant or depot formulation is constructed and arranged to deliver therapeutic or prophylactic levels of the active ingredient(s) for 120 to 180 days. In some embodiments, the implant or depot formulation is constructed and arranged to deliver therapeutic or prophylactic levels of the active ingredient(s) for up to one year. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

### III. Therapeutic Methods

### a) Inhibition of the MRGPRX2 Receptor

In one aspect, the present disclosure provides a method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with an effective amount of an inhibitor of a MRGPRX2 receptor. In some embodiments, contacting the bevemipretide injection site or intended injection site with the inhibitor of the MRGPRX2 receptor treats, prevents, ameliorates, inhibits or delays the onset of mast cell degranulation in the subject. In some embodiments, the contacting is performed by direct application to the surface of the skin at the bevemipretide injection site or intended injection site (e.g., by direct application of an ointment or cream). In some embodiments, the contacting is indirect such as by oral administration of the therapeutic agent whereby the inhibitor of the MRGPRX2 receptor is systematically applied thereby reaching many, or all, tissues of the subject, including the injection site or intended injection site.

In some embodiments, contacting the bevemipretide injection site or intended injection site with the inhibitor of the MRGPRX2 receptor arrests mast cell degranulation and the resulting immune response at the bevemipretide injection site or intended injection site of the subject. In some embodiments, contacting the injection site or intended injection site with the inhibitor of the MRGPRX2 receptor treats the bevemipretide injection site to thereby arrest mast cell degranulation and the resulting immune response at the bevemipretide injection site of the subject. In some embodiments, contacting the injection site or intended injection site with the inhibitor of the MRGPRX2 receptor prevents mast cell degranulation and the resulting immune response at the bevemipretide injection site of the subject. In some embodiments, contacting the injection site or intended injection site with the inhibitor of the MRGPRX2 receptor ameliorates mast cell degranulation and the resulting immune response at the bevemipretide injection site of the subject. In some embodiments, contacting the bevemipretide injection site with the inhibitor of the MRGPRX2 receptor inhibits the injection site reaction by arresting mast cell degranulation and the resulting immune response at the bevemipretide injection site of the subject. In some embodiments, contacting the bevemipretide injection site or intended injection site with the inhibitor of the MRGPRX2 receptor delays the onset of the injection site reaction by arresting mast cell degranulation and resulting immune response at the bevemipretide injection site of the subject.

In some embodiments, the bevemipretide injection site or intended injection site is contacted with the inhibitor of the MRGPRX2 receptor prior to administration of bevemipretide, or a pharmaceutically acceptable salt thereof. In some embodiments, the bevemipretide injection site or intended injection site is contacted with the inhibitor of the MRGPRX2 receptor after administration of bevemipretide, or a pharmaceutically acceptable salt thereof. In some embodiments, the bevemipretide injection site or intended injection site is contacted with the inhibitor of the MRGPRX2 receptor simultaneously with administration of bevemipretide, or a pharmaceutically acceptable salt thereof.

In some embodiments, the method comprises contacting the bevemipretide injection site or intended injection site with an effective amount of a flavonoid, a coumarin, a phenol or a terpenoid as the inhibitor of the MRGPRX2 receptor. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen- 1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4/,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the bevemipretide injection site or intended injection site is contacted with the flavonoid, the coumarin, the phenol or the terpenoid prior to administration of bevemipretide, or a pharmaceutically acceptable salt thereof. In some embodiments, the bevemipretide injection site or intended injection site is contacted with the flavonoid, the coumarin, the phenol or the terpenoid after administration of bevemipretide, or a pharmaceutically acceptable salt thereof. In some embodiments, the bevemipretide injection site or intended injection site is contacted with the flavonoid, the coumarin, the phenol or the terpenoid simultaneously with administration of bevemipretide, or a pharmaceutically acceptable salt thereof.

In some embodiments, the inhibitor of the MRGPRX2 receptor is mometasone furoate, tacrolimus, quercetin, and/or diphenhydramine. In some embodiments, the inhibitor is ice. Thus, in some embodiments, the method comprises contacting the bevemipretide injection site or intended injection site with mometasone furoate, tacrolimus, quercetin, diphenhydramine and/or ice. In some embodiments, the bevemipretide injection site or intended injection site is contacted with mometasone furoate, tacrolimus, quercetin, and/or diphenhydramine and/or ice prior to administration of bevemipretide, or a pharmaceutically acceptable salt thereof. In some embodiments, the bevemipretide injection site or intended injection site is contacted with mometasone furoate, tacrolimus, quercetin, and/or diphenhydramine and/or ice after administration of bevemipretide, or a pharmaceutically acceptable salt thereof. In some embodiments, the bevemipretide injection site or intended injection site is contacted with the mometasone furoate, tacrolimus, quercetin, and/or diphenhydramine and/or ice simultaneously with administration of bevemipretide, or a pharmaceutically acceptable salt thereof.

The administration of the inhibitor of the inhibitor of the MRGPRX2 receptor can be direct (e.g., applied to the area of the skin where the injection of bevemipretide is/was administered) or indirect (e.g., where the therapeutic agent is administered systematically (e.g., oral or nasal administration) and therefore is applied to the bevemipretide injection site by operation of the biological processes of the subject that directs the therapeutic agent to the skin cells in the area of the bevemipretide injection site (and elsewhere in the body of the subject). Thus, in some embodiments, the method comprises contacting the bevemipretide injection site or intended injection site with an effective amount of mometasone furoate, tacrolimus, quercetin, diphenhydramine and/or ice. In some embodiments, the mometasone furoate ointment is applied to the bevemipretide injection site or intended injection site to thereby contact the bevemipretide injection site with mometasone furoate. In some embodiments, tacrolimus ointment or quercetin ointment is applied to the bevemipretide injection site or intended injection site to thereby contact the bevemipretide injection site with tacrolimus or quercetin. In some embodiments, diphenhydramine or quercetin is administered systemically, optionally via oral administration, to the subject to thereby contact the bevemipretide injection site or intended injection site with diphenhydramine or quercetin. In some embodiments, ice is applied to the bevemipretide injection site or intended injection site to thereby contact the bevemipretide injection site with the ice.

In some embodiments of the method, the bevemipretide injection site or intended injection site is contacted with mometasone furoate ointment. In some embodiments of the method, the bevemipretide injection site or intended injection site is contacted with tacrolimus ointment. In some embodiments of the method, the bevemipretide injection site or intended injection site is contacted with quercetin, either as a topical ointment or cream or indirectly by oral administration of the quercetin. In some embodiments of the method, the bevemipretide injection site or intended injection site is contacted with tacrolimus or quercetin. In some embodiments of the method, the bevemipretide injection site or intended injection site is contacted with diphenhydramine, often indirectly/systemically by oral administration of the diphenhydramine. In some embodiments of the method, the bevemipretide injection site or intended injection site is contacted with quercetin, indirectly/systemically by oral administration of the quercetin. In some embodiments of the method, the bevemipretide injection site or intended injection site is contacted with diphenhydramine or quercetin, indirectly/systemically by oral administration of the diphenhydramine or quercetin. In some embodiments of the method, ice is applied to the bevemipretide injection site or intended injection site.

In some embodiments, the bevemipretide injection site or intended injection site is contacted with the mometasone furoate, tacrolimus, quercetin, diphenhydramine and/or ice prior to administration of bevemipretide, or a pharmaceutically acceptable salt thereof. In some embodiments, the bevemipretide injection site or intended injection site is contacted with the mometasone furoate, tacrolimus, quercetin, diphenhydramine and/or ice after administration of bevemipretide, or a pharmaceutically acceptable salt thereof. In some embodiments, the bevemipretide injection site or intended injection site is contacted with the mometasone furoate, tacrolimus, quercetin, diphenhydramine and/or ice simultaneously with administration of bevemipretide, or a pharmaceutically acceptable salt thereof.

In some embodiments of the aforementioned methods, about 5 mg to about 60 mg of bevemipretide, or a pharmaceutically acceptable salt thereof, is subcutaneously administered to the injection site. In some embodiments of the aforementioned methods, about 5 mg of bevemipretide, or a pharmaceutically acceptable salt thereof, is subcutaneously administered to the injection site. In some embodiments of the aforementioned methods, about 10 mg of bevemipretide, or a pharmaceutically acceptable salt thereof, is subcutaneously administered to the injection site. In some embodiments of the aforementioned methods, about 20 mg of bevemipretide, or a pharmaceutically acceptable salt thereof, is subcutaneously administered to the injection site. In some embodiments of the aforementioned methods, about 30 mg of bevemipretide, or a pharmaceutically acceptable salt thereof, is subcutaneously administered to the injection site. In some embodiments of the aforementioned methods, about 40 mg of bevemipretide, or a pharmaceutically acceptable salt thereof, is subcutaneously administered to the injection site. In some embodiments of the aforementioned methods, about 50 mg of bevemipretide, or a pharmaceutically acceptable salt thereof, is subcutaneously administered to the injection site. In some embodiments of the aforementioned methods, about 60 mg of bevemipretide, or a pharmaceutically acceptable salt thereof, is subcutaneously administered to the injection site.

### b) Inhibition of the Mast Cell Degranulation

In one aspect, the present disclosure provides a method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with an effective amount of an inhibitor of mast cell degranulation. In some embodiments, contacting the bevemipretide injection site or intended injection site with the inhibitor of mast cell degranulation treats, prevents, ameliorates, inhibits or delays the onset mast cell degranulation in the subject. In some embodiments, the contacting is performed by direct application to the surface of the skin at the bevemipretide injection site or intended injection site (e.g., by direct application of an ointment or cream). In some embodiments, the contacting is indirect such as by oral administration of the therapeutic agent whereby the inhibitor of the mast cell degranulation is systematically applied thereby reaching many, or all, tissues of the subject, including the injection site or intended injection site.

In some embodiments, contacting the bevemipretide injection site or intended injection site with the inhibitor of mast cell degranulation arrests mast cell degranulation and the resulting immune response at the bevemipretide injection site of the subject. In some embodiments, contacting the bevemipretide injection site or intended injection site with an inhibitor of mast cell degranulation treats the bevemipretide injection site to thereby arrest mast cell degranulation and the resulting immune response at the bevemipretide injection site of the subject. In some embodiments, contacting the injection site or intended injection site with the inhibitor of mast cell degranulation prevents mast cell degranulation and the resulting immune response at the bevemipretide injection site of the subject. In some embodiments, contacting the injection site or intended injection site with the inhibitor of mast cell degranulation ameliorates mast cell degranulation and the resulting immune response at the bevemipretide injection site of the subject. In some embodiments, contacting the injection site or intended injection site with the inhibitor of mast cell degranulation inhibits mast cell degranulation and the resulting immune response at the bevemipretide injection site of the subject. In some embodiments, contacting the injection site or intended injection site with the inhibitor of mast cell degranulation delays the onset of the injection site reaction by arresting mast cell degranulation and resulting immune response at the injection site of the subject.

In some embodiments, the bevemipretide injection site or intended injection site is contacted with the inhibitor of mast cell degranulation prior to administration of bevemipretide, or a pharmaceutically acceptable salt thereof. In some embodiments, the bevemipretide injection site or intended injection site is contacted with the inhibitor of mast cell degranulation after administration of bevemipretide, or a pharmaceutically acceptable salt thereof. In some embodiments, the bevemipretide injection site or intended injection site is contacted with the inhibitor of mast cell degranulation simultaneously with administration of bevemipretide, or a pharmaceutically acceptable salt thereof.

In some embodiments, the method comprises contacting the bevemipretide injection site or intended injection site with an effective amount of a flavonoid, a coumarin, a phenol or a terpenoid as the inhibitor of mast cell degranulation. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen- 1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the bevemipretide injection site or intended injection site is contacted with the flavonoid, the coumarin, the phenol or the terpenoid prior to administration of bevemipretide, or a pharmaceutically acceptable salt thereof. In some embodiments, the bevemipretide injection site or intended injection site is contacted with the flavonoid, the coumarin, the phenol or the terpenoid after administration of bevemipretide, or a pharmaceutically acceptable salt thereof. In some embodiments, the bevemipretide injection site or intended injection site is contacted with the flavonoid, the coumarin, the phenol or the terpenoid simultaneously with administration of bevemipretide, or a pharmaceutically acceptable salt thereof.

In some embodiments, the inhibitor of mast cell degranulation is mometasone furoate, tacrolimus, quercetin, and/or diphenhydramine. In some embodiments, the inhibitor is ice. Thus, in some embodiments, the method comprises contacting the bevemipretide injection site or intended injection site with mometasone furoate, tacrolimus, quercetin, diphenhydramine and/or ice. In some embodiments, the bevemipretide injection site or intended injection site is contacted with mometasone furoate, tacrolimus, quercetin, and/or diphenhydramine and/or ice prior to administration of bevemipretide, or a pharmaceutically acceptable salt thereof. In some embodiments, the bevemipretide injection site or intended injection site is contacted with mometasone furoate, tacrolimus, quercetin, and/or diphenhydramine and/or ice after administration of bevemipretide, or a pharmaceutically acceptable salt thereof. In some embodiments, the bevemipretide injection site or intended injection site is contacted with the mometasone furoate, tacrolimus, quercetin, and/or diphenhydramine and/or ice simultaneously with administration of bevemipretide, or a pharmaceutically acceptable salt thereof.

In some embodiments, contacting the bevemipretide injection site or intended injection site with the inhibitor or mast cell degranulation can be can be direct (e.g., applied to the area of the skin where the injection is/was administered) or indirect (e.g., where the inhibitor or mast cell degranulation is administered systematically (e.g., oral or nasal administration) and therefore is applied to the bevemipretide injection site or intended injection site by operation of the biological processes of the subject that directs the therapeutic agent to the skin cells in the area of the bevemipretide injection site (and elsewhere in the body of the subject). Thus, in some embodiments, mometasone furoate ointment is applied to the injection site or intended injection site to thereby contact the bevemipretide injection site with mometasone furoate. In some embodiments, tacrolimus ointment or quercetin ointment is applied to the injection site or intended injection site to thereby contact the bevemipretide injection site with tacrolimus or quercetin. In some embodiments, diphenhydramine or quercetin is administered systemically, optionally via oral administration, to the subject to thereby contact the bevemipretide injection site or intended injection site with diphenhydramine or quercetin. In some embodiments, ice is applied to the injection site or intended injection site to thereby contact the bevemipretide injection site with the ice.

In some embodiments of the method, the bevemipretide injection site or intended injection site is contacted with mometasone furoate ointment. In some embodiments of the method, the bevemipretide injection site or intended injection site is contacted with tacrolimus ointment. In some embodiments of the method, the bevemipretide injection site or intended injection site is contacted with quercetin, either as a topical ointment or cream or indirectly by oral administration of the quercetin. In some embodiments of the method, the bevemipretide injection site or intended injection site is contacted with tacrolimus or quercetin. In some embodiments of the method, the bevemipretide injection site or intended injection site is contacted with diphenhydramine, often indirectly/systemically by oral administration of the diphenhydramine. In some embodiments of the method, the bevemipretide injection site or intended injection site is contacted with quercetin, indirectly/systemically by oral administration of the quercetin. In some embodiments of the method, the bevemipretide injection site or intended injection site is contacted with diphenhydramine or quercetin, indirectly/systemically by oral administration of the diphenhydramine or quercetin. In some embodiments of the method, ice is applied to the bevemipretide injection site or intended injection site.

In some embodiments, the bevemipretide injection site or intended injection site is contacted with the mometasone furoate, tacrolimus, quercetin, diphenhydramine and/or ice prior to administration of bevemipretide, or a pharmaceutically acceptable salt thereof. In some embodiments, the bevemipretide injection site or intended injection site is contacted with the mometasone furoate, tacrolimus, quercetin, diphenhydramine and/or ice after administration of bevemipretide, or a pharmaceutically acceptable salt thereof. In some embodiments, the bevemipretide injection site or intended injection site is contacted with the mometasone furoate, tacrolimus, quercetin, diphenhydramine and/or ice simultaneously with administration of bevemipretide, or a pharmaceutically acceptable salt thereof.

In some embodiments of the aforementioned methods, about 5 mg to about 60 mg of bevemipretide, or a pharmaceutically acceptable salt thereof, is subcutaneously administered to the injection site. In some embodiments of the aforementioned methods, about 5 mg of bevemipretide, or a pharmaceutically acceptable salt thereof, is subcutaneously administered to the injection site. In some embodiments of the aforementioned methods, about 10 mg of bevemipretide, or a pharmaceutically acceptable salt thereof, is subcutaneously administered to the injection site. In some embodiments of the aforementioned methods, about 20 mg of bevemipretide, or a pharmaceutically acceptable salt thereof, is subcutaneously administered to the injection site. In some embodiments of the aforementioned methods, about 30 mg of bevemipretide, or a pharmaceutically acceptable salt thereof, is subcutaneously administered to the injection site. In some embodiments of the aforementioned methods, about 40 mg of bevemipretide, or a pharmaceutically acceptable salt thereof, is subcutaneously administered to the injection site. In some embodiments of the aforementioned methods, about 50 mg of bevemipretide, or a pharmaceutically acceptable salt thereof, is subcutaneously administered to the injection site. In some embodiments of the aforementioned methods, about 60 mg of bevemipretide, or a pharmaceutically acceptable salt thereof, is subcutaneously administered to the injection site.

### c) Applying a Therapeutic Agent to the Injection Site

In one aspect, the present disclosure provides a method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with mometasone furoate, tacrolimus, quercetin, diphenhydramine and/or ice. In some embodiments, contacting the bevemipretide injection site or intended injection site with mometasone furoate, tacrolimus, quercetin, diphenhydramine and/or ice treats, prevents, ameliorates, inhibits or delays the onset mast cell degranulation in the subject. In some embodiments, contacting the bevemipretide injection site or intended injection site with mometasone furoate, tacrolimus, quercetin, diphenhydramine and/or ice treats the bevemipretide injection site to thereby arrest mast cell degranulation and the resulting immune response at the bevemipretide injection site of the subject. In some embodiments, contacting the bevemipretide injection site or intended injection site with mometasone furoate, tacrolimus, quercetin, diphenhydramine and/or ice prevents mast cell degranulation and the resulting immune response at the bevemipretide injection site of the subject. In some embodiments, contacting the bevemipretide injection site or intended injection site with mometasone furoate, tacrolimus, quercetin, diphenhydramine and/or ice ameliorates mast cell degranulation and the resulting immune response at the bevemipretide injection site of the subject. In some embodiments, contacting the bevemipretide injection site or intended injection site with mometasone furoate, tacrolimus, quercetin, diphenhydramine and/or ice inhibits mast cell degranulation and the resulting immune response at the bevemipretide injection site of the subject. In some embodiments, contacting the bevemipretide injection site or intended injection site with mometasone furoate, tacrolimus, quercetin, diphenhydramine and/or ice delays the onset the injection site reaction by arresting mast cell degranulation and the resulting immune response at the bevemipretide injection site of the subject.

In some embodiments, the bevemipretide injection site or intended injection site can be contacted with the mometasone furoate, tacrolimus, quercetin, diphenhydramine and/or ice prior to administration of bevemipretide, or a pharmaceutical salt thereof. In some embodiments, the bevemipretide injection site or intended injection site can be contacted with the mometasone furoate, tacrolimus, quercetin, diphenhydramine and/or ice after administration of bevemipretide, or a pharmaceutical salt thereof. In some embodiments, the bevemipretide injection site or intended injection site can be contacted with the mometasone furoate, tacrolimus, quercetin, diphenhydramine and/or ice simultaneously with administration of bevemipretide, or a pharmaceutical salt thereof.

Thus, in some embodiments, mometasone furoate ointment is applied to the injection site or intended injection site to thereby contact the bevemipretide injection site with mometasone furoate. In some embodiments, tacrolimus ointment or quercetin ointment is applied to the injection site or intended injection site to thereby contact the bevemipretide injection site with tacrolimus or quercetin. In some embodiments, diphenhydramine or quercetin is administered systemically, optionally via oral administration, to the subject to thereby contact the bevemipretide injection site or intended injection site with diphenhydramine or quercetin. In some embodiments, ice is applied to the injection site or intended injection site to thereby contact the bevemipretide injection site with the ice.

In some embodiments, contacting the bevemipretide injection site or intended injection site with the mometasone furoate, tacrolimus, quercetin, diphenhydramine and/or ice can be can be direct (e.g., applied to the area of the skin where the injection is/was administered) or indirect (e.g. where the inhibitor or mast cell degranulation is administered systematically (e.g., oral or nasal administration) and therefore is applied to the bevemipretide injection site by operation of the biological processes of the subject that directs the therapeutic agent to the skin cells in the area of the bevemipretide injection site (and elsewhere in the body of the subject).

In some embodiments of the method, the bevemipretide injection site or intended injection site is contacted with mometasone furoate ointment. In some embodiments of the method, the bevemipretide injection site or intended injection site is contacted with tacrolimus ointment. In some embodiments of the method, the bevemipretide injection site or intended injection site is contacted with quercetin, either as a topical ointment or cream or indirectly by oral administration of the quercetin. In some embodiments of the method, the bevemipretide injection site or intended injection site is contacted with tacrolimus or quercetin. In some embodiments of the method, the bevemipretide injection site or intended injection site is contacted with diphenhydramine, often indirectly/systemically by oral administration of the diphenhydramine. In some embodiments of the method, the bevemipretide injection site or intended injection site is contacted with quercetin, indirectly/systemically by oral administration of the quercetin. In some embodiments of the method, the bevemipretide injection site or intended injection site is contacted with diphenhydramine or quercetin, indirectly/systemically by oral administration of the diphenhydramine or quercetin. In some embodiments of the method, ice is applied to the bevemipretide injection site or intended injection site.

In some embodiments of the aforementioned methods, about 5 mg to about 60 mg of bevemipretide, or a pharmaceutically acceptable salt thereof, is subcutaneously administered to the injection site. In some embodiments of the aforementioned methods, about 5 mg of bevemipretide, or a pharmaceutically acceptable salt thereof, is subcutaneously administered to the injection site. In some embodiments of the aforementioned methods, about 10 mg of bevemipretide, or a pharmaceutically acceptable salt thereof, is subcutaneously administered to the injection site. In some embodiments of the aforementioned methods, about 20 mg of bevemipretide, or a pharmaceutically acceptable salt thereof, is subcutaneously administered to the injection site. In some embodiments of the aforementioned methods, about 30 mg of bevemipretide, or a pharmaceutically acceptable salt thereof, is subcutaneously administered to the injection site. In some embodiments of the aforementioned methods, about 40 mg of bevemipretide, or a pharmaceutically acceptable salt thereof, is subcutaneously administered to the injection site. In some embodiments of the aforementioned methods, about 50 mg of bevemipretide, or a pharmaceutically acceptable salt thereof, is subcutaneously administered to the injection site. In some embodiments of the aforementioned methods, about 60 mg of bevemipretide, or a pharmaceutically acceptable salt thereof, is subcutaneously administered to the injection site.

These doses of bevemipretide, or a pharmaceutically acceptable salt thereof, according to any of the methods described in sections III (a) to III(c), above, can be administered once daily to the subject for as long as administration results in beneficial effects on the subject with respect to the disease, disorder or condition being addressed. Alternatively, lesser amounts can be administered several times per day so that that total daily dose is the same as with a single daily dose referred to above.

### d) Method for Administering Bevemipretide

In one aspect, the present disclosure provides a method comprising: (a) subcutaneously administering an effective amount of bevemipretide, or a pharmaceutically acceptable salt thereof, to a subject; and (b) also administering to said subject an inhibitor of a MRGPRX2 receptor and/or inhibitor of mast cell degranulation, wherein steps (a) and (b) can be performed in either order or simultaneously. In some embodiments, the subject is administered an inhibitor of a MRGPRX2 receptor. In some embodiments, step (a) is performed prior to performing step (b). In some embodiments, step (b) is performed prior to performing step (a). In some embodiments, step (a) and step (b) are performed simultaneously or substantially simultaneously. In some embodiments, the subject is administered an inhibitor of mast cell degranulation. In some embodiment of the foregoing methods, mometasone furoate, tacrolimus, quercetin, diphenhydramine and/or ice is administered to the subject as an inhibitor of a MRGPRX2 receptor and/or inhibitor of mast cell degranulation. In some embodiment, the subject is human.

In some embodiments of the aforementioned methods, about 5 mg to about 60 mg of bevemipretide, or a pharmaceutically acceptable salt thereof, is subcutaneously administered to the injection site. In some embodiments of the aforementioned methods, about 5 mg of bevemipretide, or a pharmaceutically acceptable salt thereof, is subcutaneously administered to the injection site. In some embodiments of the aforementioned methods, about 10 mg of bevemipretide, or a pharmaceutically acceptable salt thereof, is subcutaneously administered to the injection site. In some embodiments of the aforementioned methods, about 20 mg of bevemipretide, or a pharmaceutically acceptable salt thereof, is subcutaneously administered to the injection site. In some embodiments of the aforementioned methods, about 30 mg of bevemipretide, or a pharmaceutically acceptable salt thereof, is subcutaneously administered to the injection site. In some embodiments of the aforementioned methods, about 40 mg of bevemipretide, or a pharmaceutically acceptable salt thereof, is subcutaneously administered to the injection site. In some embodiments of the aforementioned methods, about 50 mg of bevemipretide, or a pharmaceutically acceptable salt thereof, is subcutaneously administered to the injection site. In some embodiments of the aforementioned methods, about 60 mg of bevemipretide, or a pharmaceutically acceptable salt thereof, is subcutaneously administered to the injection site.

In practice of any of the aforementioned methods disclosed in sections III(a) to III(d), above, in some embodiments, the subject has been diagnosed with amyotrophic lateral sclerosis (ALS).

In practice of any of the aforementioned methods disclosed in sections III(a) to III(d), above, in some embodiments, the subject has been diagnosed with Parkinson's disease (PD).

In practice of any of the aforementioned methods disclosed in sections III(a) to III(d), above, in some embodiments, the subject has been diagnosed with PD with dementia.

In practice of any of the aforementioned methods disclosed in sections III(a) to III(d), above, in some embodiments, the subject has been diagnosed with dementia with Lewy bodies.

In practice of any of the aforementioned methods disclosed in sections III(a) to III(d), above, in some embodiments, the subject has been diagnosed with Multiple System Atrophy.

In practice of any of the aforementioned methods disclosed in sections III(a) to III(d), above, in some embodiments, the subject has been diagnosed with Frontotemporal Lobar Degeneration (FTLD).

In practice of any of the aforementioned methods disclosed in sections III(a) to III(d), above, in some embodiments, the subject has been diagnosed with a disease where TDP-43, Tau protein and α-synuclein are associated with the disease pathology.

In practice of any of the aforementioned methods disclosed in sections III(a) to III(d), above, in some embodiments, the subject has been diagnosed with diabetic macular edema.

In practice of any of the aforementioned methods disclosed in sections III(a) to III(d), above, in some embodiments, the subject has been diagnosed with macular degeneration.

In practice of any of the aforementioned methods disclosed in sections III(a) to III(d), above, in some embodiments, the subject has been diagnosed with wet age-related macular degeneration or dry age-related macular degeneration.

In practice of any of the aforementioned methods disclosed in sections III(a) to III(d), above, in some embodiments, the subject has been diagnosed with wet age-related macular degeneration or dry age-related macular degeneration with drusen.

In practice of any of the aforementioned methods disclosed in sections III(a) to III(d), above, in some embodiments, the subject has been diagnosed with wet age-related macular degeneration or dry age-related macular degeneration with geographic atrophy (GA).

In practice of any of the aforementioned methods disclosed in sections III(a) to III(d), above, in some embodiments, the subject has been diagnosed with glaucoma.

In practice of any of the aforementioned methods disclosed in sections III(a) to III(d), above, in some embodiments, the subject has been diagnosed with diabetic retinopathy.

In practice of any of the aforementioned methods disclosed in sections III(a) to III(d), above, in some embodiments, the subject has been diagnosed with retinitis pigmentosa.

### EXAMPLES

The present technology is further illustrated by the following examples, which should not be construed as limiting in any way.

### Example 1—Injection Site Reactions Induced by Bevemipretide are Mediated by Activation of Mast Cells Through the G Protein-Coupled Receptor MRGPRX2

Summary: This study was conducted to demonstrate the feasibility of preparing an in vitro assay to determine whether or not bevemipretide activates the human MRGPRX2 receptor and thereby induce mast cell degranulation as well as provide a possible screening assay to determine the efficacy of potential inhibitors of the activation of the human MRGPRX2 receptor.

### Experimental Design

### EC50 Value for MRGPRX2

A clonal HEK293 cell line stably expressing human MRGPRX2 and Galpha15 was used for the assay, which was performed using a fluorescent plate reader. Cells were plated at 40,000 cells per well in a glass-bottom 96 well plate 20 hours before the assay. Cells were loaded with N-[4-[6-[(Acetyloxy)methoxy]-2,7-difluoro-3-oxo-3H-xanthen-9-yl]-2-[2-[2-[bis[2-[(acetyloxy)methoxy]-2-oxoethyl]amino]-5-methylphenoxy]ethoxy]phenyl]-N-[2-[(acetyloxy)methoxy]-2-oxoethyl]glycine (acetyloxy)methyl ester (Fluo-4 AM) for 45 minutes at 37°° C. and allowed to rest for 30 minutes before use. Baseline fluorescence was calculated as the average of a 30 second read, with data points taken at 5 second intervals. Response was defined as the maximum signal within 90 seconds after addition of bevemipretide, subtracted by the baseline fluorescence signal. Concentrations were tested in duplicate and the assay was run 6 times. The curve was calculated as a four-parameter non-linear fit with variable slope. Traces were obtained using the same cell line and same Fluo-4 AM loading protocol but were imaged using a fluorescence microscope using a standard fluorescein isothiocyanate (FITC) filter. Fluorescence for different cells was calculated using region of interest selections.

### Confirmation of Mast Cell Activation and Inhibition

MRGPRX2 expressing and control (non-MRGPRX2 expressing) Chem-1 cells lines were obtained from Discovery Services, EuroFins. Cells were cultured in Dulbecco's Modified Eagle Medium (DMEM) high glucose medium (4.5 g/L) with 10% fetal bovine serum (FBS), non-essential amino acids, and N-2-hydroxyethylpiperazine-N′-2-ethanesulfonic acid (HEPES) buffer (pH 7.0-7.6). Geneticin (G418) was used for selection of MRGPRX2-expressing Chem-1 cells. Cells were maintained at 37° C. at 5% CO₂. Cells were seeded to a glass bottom 96-well plates at a density of 50,000 per well (Cellvis). Cells were loaded with Fluo-4 AM for 30 minutes at 37° C. at a concentration of 2.5 μM. Cells were washed once with Dulbecco's phosphate buffered saline (DPBS). Cells were rested at room temperature for 30 minutes before use and washed with DPBS. Cells were resuspended in 50 μL phosphate buffered saline (PBS) with calcium and magnesium. To stimulate cells, 50 μL of 2× drug (bevemipretide or leuprolide) or DPBS as a control. Plates were read immediately with a Biotek plate reader. Readings were taken every 6 seconds for 2 minutes.

This assay was designed as a calcium-based readout of receptor activity. As such, the assay can be used to determine if a test article will elicit an immune response associated with the MRGPRX2 receptor, as well as possibly demonstrate the efficacy of inhibitors of the receptor.

### Results

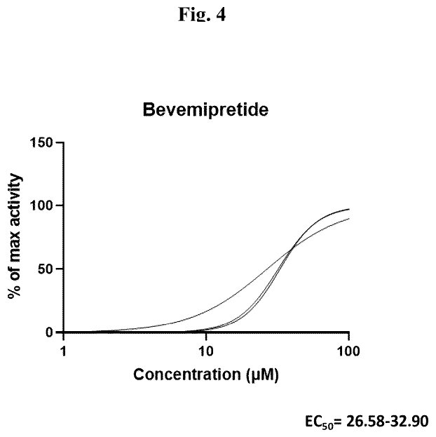

Using a fluorescence plate reader and multiple runs, it was determined that the EC50 for MRGPRX2 was 26.6-32.9 micrograms/mL (FIG. 4).

With reference to FIG. 5A and FIG. 5B, the results demonstrate that bevemipretide exhibits a dose dependent activation of the MRGPRX2 expressing cells (FIG. 5A) but no effect on cells lacking the MRGPRX2 receptor (FIG. 5B). Ionomycin is a positive control that generates a calcium response regardless of the presence or absence of the MRGPRX2 receptor (the parent Chem-1 cells lack the MRGPRX2 receptor) and thereby confirms that the cells of the assay are responsive to calcium flux.

With reference to FIG. 6, the results demonstrate that leuprolide exhibits a dose dependent activation of the MRGPRX2 expressing cells. Leuprolide is an expected activator of the MRGPRX2 receptor and therefore confirms the expected function of the assay when an activator of the MRGPRX2 receptor is present. In this way, the interpretation of the results of FIG. 5A and FIG. 5B are further confirmed.

With reference to FIGS. 7A and 7B, the results indicate that quercetin or monetasone can, in a dose-dependent manner, inhibit the activation of the MRGPRX2 receptor that is caused by the presence of bevemipretide. As such, the results suggest that this assay can be used to screen possible inhibitors suitable to treat, prevent, ameliorate, inhibit or delay the onset of injection site reactions caused by subcutaneous injection of bevemipretide in a subject.

Conclusions: The data in this Example 3 support the following conclusions:
Bevemipretide activates the human MRGPRX2 receptor;
It is possible to inhibit bevemipretide-mediated activation of MRGPRX2;
This in vitro assay can be used to screen possible inhibitors of the activation of the human MRGPRX2 receptor; and
Quercetin and monetasone are inhibitors of the activation of the human MRGPRX2 receptor.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with an effective amount of an inhibitor of a MRGPRX2 receptor.

2. The method of claim 1, wherein contacting the bevemipretide injection site or intended injection site with the inhibitor of the MRGPRX2 receptor treats, prevents, ameliorates, inhibits or delays the onset of mast cell degranulation in the subject.

3. The method of claim 1, wherein the method comprises contacting the bevemipretide injection site or intended injection site with an effective amount of a flavonoid, a coumarin, a phenol or a terpenoid as the inhibitor of a MRGPRX2 receptor.

4. The method of claim 3, wherein the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin.

5. The method of claim 3, wherein the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione).

6. The method of claim 3, wherein the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one).

7. The method of claim 3, wherein the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

8. The method of claim 3, wherein the bevemipretide injection site or intended injection site is contacted with the flavonoid, the coumarin, the phenol or the terpenoid prior to administration of bevemipretide, or a pharmaceutically acceptable salt thereof.

9. The method of claim 3, wherein the bevemipretide injection site or intended injection site is contacted with the flavonoid, the coumarin, the phenol or the terpenoid after administration of bevemipretide, or a pharmaceutically acceptable salt thereof.

10. The method of claim 1, wherein the method comprises contacting the bevemipretide injection site or intended injection site with an effective amount of mometasone furoate, tacrolimus, quercetin, diphenhydramine and/or ice.

11. The method of claim 10, wherein mometasone furoate ointment is applied to the injection site to thereby contact the bevemipretide injection site or intended injection site with mometasone furoate.

12. The method of claim 10, wherein tacrolimus ointment or quercetin ointment is applied to the injection site to thereby contact the bevemipretide injection site or intended injection site with tacrolimus or quercetin.

13. The method of claim 10, wherein diphenhydramine or quercetin is administered systemically, optionally via oral administration, to the subject to thereby contact the bevemipretide injection site or intended injection site with diphenhydramine or quercetin.

14. The method of claim 10, wherein ice is applied to the injection site or intended injection site to thereby contact the bevemipretide injection site or intended injection site with the ice.

15. The method of claim 1, wherein the bevemipretide injection site or intended injection site is contacted with the mometasone furoate, tacrolimus, quercetin, diphenhydramine and/or ice prior to administration of bevemipretide, or a pharmaceutically acceptable salt thereof.

16. The method of claim 1, wherein the bevemipretide injection site or intended injection site is contacted with the mometasone furoate, tacrolimus, quercetin, diphenhydramine and/or ice after administration of bevemipretide, or a pharmaceutically acceptable salt thereof.

17. The method of claim 1, wherein about 5 mg to about 60 mg of bevemipretide, or a pharmaceutically acceptable salt thereof, is subcutaneously administered to the bevemipretide injection site.

18. A method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with an effective amount of an inhibitor of mast cell degranulation.

19-33. (canceled)

34. A method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with a flavonoid, a coumarin, a phenol or a terpenoid.

35-41. (canceled)

42. A method for treating, preventing, ameliorating, inhibiting or delaying the onset of injection site reactions associated with subcutaneous injections of bevemipretide, or a pharmaceutically acceptable salt thereof, administered to a subject in need thereof, comprising contacting a bevemipretide injection site or intended injection site with mometasone furoate, tacrolimus, quercetin, diphenhydramine and/or ice.

43-56. (canceled)

57. A method comprising:
a) subcutaneously administering an effective amount of bevemipretide, or a pharmaceutically acceptable salt thereof, to a subject in need thereof; and
b) also administering to said subject an inhibitor of a MRGPRX2 receptor and/or inhibitor of mast cell degranulation;
wherein steps a) and b) can be performed in either order or simultaneously.

58. The method of claim 57, wherein step (a) is performed prior to performing step (b).

59. The method of claim 57, wherein step (b) is performed prior to performing step (a).

60. The method of claim 57, wherein step (a) and step (b) are performed simultaneously or substantially simultaneously.

61-77. (canceled)

78. The method of claim 60, wherein the subject has been diagnosed as having age-related macular degeneration (AMD).

79. The method of claim 78, wherein the subject has drusen.

80. (canceled)

81. The method of claim 1, wherein the subject has been diagnosed with amyotrophic lateral sclerosis (ALS), Parkinson's disease (PD), PD with dementia, dementia with Lewy bodies, Multiple Systems Atrophy, Frontal Lobar Degeneration or other disease where TDP-43, Tau protein and α-synuclein are associated with the disease pathology.
